(19) United States
(12) Patent Application Publication
(10) Pub. No.: US 2025/0161507 A1
(43) Pub. Date: May 22, 2025

(54) Sanitizing Scan Device

(71) Applicants: James Hatchett, San Antonio, TX (US); Jeffrey Hatchett, San Antonio, TX (US)

(72) Inventors: James Hatchett, San Antonio, TX (US); Jeffrey Hatchett, San Antonio, TX (US)

(21) Appl. No.: 18/430,777

(22) Filed: Feb. 2, 2024

Related U.S. Application Data
(60) Provisional application No. 63/601,256, filed on Nov. 21, 2023.

(51) Int. Cl. A61L 2/10

(52) U.S. Cl. CPC A61L 2/10; A61L 2202/11; A61L 2202/14

(57) ABSTRACT

A sanitizing scan device is disclosed that utilizes ultraviolet (UV) light for eliminating germs, bacteria, and viruses from different surfaces. In use, the UV light is refracted and covers the surface area to be sanitized. The sanitizing scan device comprises a housing and cover that is a durable, weather, insect, and water-proof gasket-sealed system. Further, the housing comprises a motion-operated sensor mounted near the opening to the housing. The sensor recognizes the approach of a user's hand and acts to open the cover for use. Further, size and configuration details of the housing would vary according to application, but examples include gas pump handles, public bathroom stall handles, public hand dryers, light switches, residential and commercial entryways, and exits, appliances, vehicle handles, etc.

### CROSS-REFERENCE TO RELATED APPLICATION

The present application claims priority to, and the benefit of, U.S. Provisional Application No. 63/601,256, which was filed on Nov. 21, 2023, and is incorporated herein by reference in its entirety.

### FIELD OF THE INVENTION

The present invention relates generally to the field of sanitizing scan devices. More specifically, the present invention relates to a durable, weatherproof covering equipped with ultraviolet light sanitizing capabilities that can be installed over numerous items and areas. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in sanitizing scan devices. Generally, high traffic public areas like offices, retail stores, public restrooms, public transportation, etc., must be constantly cleaned to maintain sanitary conditions. Accordingly, without regular cleaning, a significant spread of germs and bacteria can occur. However, manually wiping down these surfaces each day may not be possible, ultimately leading to the spread of serious illness. Further, chemicals and other sanitary products can be harmful to the environment and cause rashes on the skin for some people.

Generally, various harmful pathogens, germs, bacteria, viruses, and the like are present in the environment and are responsible for the spread of infectious diseases amongst people. More specifically, such harmful pathogens can easily transmit from one person to another and may cause serious illness and even death in critical cases. These harmful pathogens may be present on any surface touched by an infected person, which when touched by a healthy person, leads to the spread of diseases amongst individuals. When an infected person sneezes, coughs, or even touches a surface, the bacteria, viruses, and other disease-causing microbes adhere to said surfaces after contact. In this manner, it is believed that billions of individuals have spread infectious diseases among each other over the years including, without limitation, the common cold, influenza, rotavirus, hepatitis A, tuberculosis, conjunctivitis, staphylococcal bacterial infections, COVID-19, strep throat and other streptococcal bacterial infections.

Proper and preventative sanitization requires an individual to manually wipe and clean each and every surface that he or she comes into contact with a disinfecting or sanitizing solution. However, manually cleaning every such surface is time consuming, tiring, ineffective, and may result in some spots being left unclean. This can lead to the spread of germs, bacteria, and dangerous viruses. Also, with manual cleaning and sanitizing attempts, it is also difficult to ensure high-level disinfection due to a number of challenges including, but not limited to, missed areas, re-contamination from dirty sponges and mops, and improper usage and mixing of antibacterial cleaning solutions, etc.

Accordingly, there is a demand for a sanitizing scan device that may be used to effectively and automatically disinfect contact surfaces as per the desires of the user, and that can eliminate the disease-causing microbes commonly found on frequently touched surfaces. More particularly, there is a demand for a sanitizing scan device that can be easily and automatically controlled by the user, and that prevents the transmission of harmful pathogens and therefore prevents the spread of infectious diseases.

Therefore, there exists a long-felt need in the art for a sanitizing scan device that provides users with a durable, weatherproof covering equipped with ultraviolet light sanitizing capabilities that can be installed over numerous items and areas. There is also a long-felt need in the art for a sanitizing scan device that allows the UV lights to kill germs, viruses, and bacteria prior to anyone touching the surface to help prevent the spread of illness. Further, there is a long-felt need in the art for a sanitizing scan device that reflects UV light within the system to fully cleanse the entire area of harmful bacteria. Moreover, there is a long-felt need in the art for a device that includes a motion sensor that expands and retracts a cover over the surface, ensuring it is only accessible when a person is ready to touch or use that surface. Further, there is a long-felt need in the art for a sanitizing scan device that improves public safety while reducing the amount of manual labor required to clean different high traffic surfaces. Finally, there is a long-felt need in the art for a sanitizing scan device that is powered by an internal battery or transformer for larger applications.

The subject matter disclosed and claimed herein, in one embodiment thereof, comprises a sanitizing scan device. The device is a sanitation device that utilizes ultraviolet (UV) light for eliminating germs, bacteria, and viruses from different surfaces. In use, the UV light is refracted and covers the surface area to be sanitized. The sanitizing scan device comprises a housing and cover that is a durable, weather, insect, and water-proof gasket-sealed system. The housing is made from a durable plastic or other suitable material that also conceals the UV-C light during operation. The interior of the device is coated with reflective durable materials that will ensure UV light covers the entire surface area within the housing. Further, the cover comprises a motion-operated sensor mounted near the opening to the housing. The sensor recognizes the approach of a user's extremity and acts to open the cover. Further, size and configuration details of the housing would vary according to application, but examples include gas pump handles, public bathroom stall handles, public hand dryers, light switches, residential and commercial entryways, and exits, appliances, vehicle handles, etc. Additionally, based on application size, the device is powered by an internal, rechargeable battery or transformer for larger applications.

In this manner, the sanitizing scan device of the present invention accomplishes all of the foregoing objectives and provides users with a device that utilizes UV light to kill germs on a surface. The device improves public safety while reducing the manual labor required to clean surfaces. The device can be powered by batteries.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a sanitizing scan device. The device is a sanitation device that utilizes ultraviolet (UV) light for eliminating germs, bacteria, and viruses from different surfaces. In use, the UV light is refracted and covers the surface area to be sanitized. The sanitizing scan device comprises a housing and cover that is a durable, weather, insect, and water-proof gasket-sealed system. Further, size and configuration details of the housing would vary according to application, but examples include gas pump handles, public bathroom stall handles, public hand dryers, light switches, residential and commercial entryways, and exits, appliances, vehicle handles, etc. Additionally, based on application size, the device is powered by an internal, rechargeable battery or transformer for larger applications.

In one embodiment, the sanitizing scan device is a UV light sanitization device that may be used to effectively and automatically disinfect contact surfaces, objects, and the like, and that can eliminate the disease-causing microbes commonly found thereon. Additionally, the sanitizing scan device is easily and automatically controlled by a user and prevents the transmission of harmful pathogens and therefore prevents the spread of infectious diseases. Moreover, the sanitizing scan device is portable and relatively maintenance free, inexpensive to manufacture and operate, and is both safe and easy to use.

In one embodiment, the sanitizing scan device comprises a housing with a cover and a plurality of ultraviolet radiating sources contained therein that emit light at the proper wavelength suitable for sanitizing the surrounding area. The emitted germicidal light is refracted and covers the surface area to be sanitized. Specifically, the interior of the housing is coated with a reflective durable material that will ensure the UV light covers the entire surface area within the housing.

In one embodiment, the interior sidewalls of the housing include reflective material to redirect ultraviolet light from the ultraviolet-radiating sources. The reflective material can be made of a thin polished layer of metal, such as aluminum, which is deposited on glass substrates. Other reflective materials may include, but are not limited to, silver and gold. Silver, which has a reflectivity of about ninety-five percent to about ninety-nine percent can reflect ultraviolet spectral regions. Gold, which has a reflectivity of about ninety-eight percent to about ninety-nine percent, can reflect light which has wavelengths below five hundred fifty nanometers. Increasing and decreasing the density and thickness of the metals used also determines the reflectivity of the sidewalls. Preferably, the metals are grounded.

In one embodiment, instead of using reflective metals as described above, reflective coatings can be placed on the sidewalls. In exemplary embodiments, dielectric coatings having a different refractive index to the substrate may be used. Dielectric coatings can include magnesium fluoride, calcium fluoride, and various metal oxides, which are deposited onto a substrate. By carefully choosing the exact composition, thickness, and number of layers, reflectivity and transmitivity of the coating can produce any desired characteristic.

In one embodiment, the cover is preferably sealed to the housing around the periphery via a gasket seal and the rear surface of the housing has an adhesive tape, or other means, to mount the housing around any surface or object to be sanitized. Alternatively, the rear surface may include slotted holes for mounting the device to a wall using one or more screws or other fasteners. The adhesive tape at the rear surface allows the scan device to removably attach to a surface and thus increases the utility of the scan device. Thus, the sanitizing scan device provides effective protection from germs, viruses, bacteria, dust mites, and mold without the use of aerosol sprays or other harsh chemicals that may be harmful if inhaled by an individual, particularly someone with a respiratory illness.

In one embodiment, the ultraviolet radiating sources can be high intensity ultraviolet lamps, ultraviolet light emitting diodes (LEDs), super luminescent LEDs, or a combination thereof. The UV radiating sources are preferably UV-C, having a significant portion of light output in the region of 100 nm to 400 nm wavelength, preferably 200 to 300 nm wavelength, more preferably 250 nm to 260 nm wavelength. Further, ultraviolet light sources can also be interchanged to provide additional types of radiation to the item. Typically, ultraviolet light sources can produce light having wavelengths from about one hundred nanometers to about four hundred nanometers. More preferably, however, ultraviolet light having wavelengths of about two hundred fifty nanometers have been used to eliminate microbes. In one embodiment, pulsating ultraviolet light sources may be used. Experiments have shown that pulsating ultraviolet light sources more effectively kill bacteria and other microbes that affect the items to be sanitized. In preferred embodiments, ultraviolet light sources can be made from quartz and a material called soft glass.

In one embodiment, the housing is configured in a cylindrical, oval, square, or any other suitable shape as is known in the art, depending on the application. Typically, the housing has an open back and a perimeter sidewall with an open front, creating a cavity for encompassing the object to be sanitized. The open back of the housing is placed around the object to be sanitized and secured in place via any suitable securing means. The cover of the device is then sealed to the front of the housing, completely enclosing the object. The housing and cover are made from a durable plastic or other suitable material that also conceals the UV-C light during operation. The interior of the housing and cover are also coated with a reflective durable material that will ensure UV light covers the entire interior surface area within the housing and cover, to completely sanitize the enclosed object.

In one embodiment, the housing comprises a motion sensor mounted near the front opening. The motion sensor acts to recognize the approach of a user's hand or other body part. Once the motion sensor recognizes the user's hand, the cover is automatically opened to allow access to the interior of the housing, and thus utilize the light switch or other object contained within. Accordingly, the cover can be secured to the housing via a movable hinge, telescoping cover, sliding cover, etc. Thus, the motion sensor expands and retracts the cover over the housing, ensuring the interior of the housing is only accessible when a person is ready to touch or use the sanitized surface.

In one embodiment, based on application size, the device is powered via an internal, rechargeable battery or transformer for larger applications. Example applications include, but are not limited to, gas pump handles, public bathroom stall handles, public hand dryers, light switches, residential and commercial entryways and exits, appliances, vehicle handles, etc., or any other suitable objects or areas as is known in the art.

In one embodiment, the device can be controlled by sending an instruction from the user through a remote device, such as a smartphone, tablet, smart watch, computer, or the like. Thus, the sanitizing scan device may be controlled wirelessly through a remote or electronic device.

In yet another embodiment, the sanitizing scan device comprises a plurality of indicia.

In yet another embodiment, a method of sanitizing a high-use item with UV lights to kill germs, viruses, and bacteria is disclosed. The method includes the steps of providing a sanitizing scan device comprising a housing and cover with UV lights positioned in the interior of the housing for sanitation. The method also comprises positioning the housing over an item to be sanitized, such as a light switch. Further, the method comprises securing the cover over the housing and light switch. The method also comprises sanitizing the light switch via the UV light within the housing. Finally, the method comprises sensing a user's hand via the motion sensor to open the cover and expose the light switch for use.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the sanitizing scan device of the present invention showing how a motion sensor activates when a hand is placed in front of the device in accordance with the disclosed architecture;

FIG. 2 illustrates a perspective view of one embodiment of the sanitizing scan device of the present invention showing how the cover is retracted during use in accordance with the disclosed architecture;

FIG. 3 illustrates a perspective view of one embodiment of the sanitizing scan device of the present invention showing the light switch protected by the device in accordance with the disclosed architecture;

FIG. 4 illustrates a perspective view of one embodiment of the sanitizing scan device of the present invention showing how a device is installed over a public restroom stall door handle in accordance with the disclosed architecture;

FIG. 5 illustrates a perspective view of one embodiment of the sanitizing scan device of the present invention showing the device sanitizing a public restroom stall door handle in accordance with the disclosed architecture; and

FIG. 6 illustrates a flowchart showing the method of sanitizing a high-use item with UV lights to kill germs, viruses, and bacteria in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a sanitizing scan device that provides users with a durable, weatherproof covering equipped with ultraviolet light sanitizing capabilities that can be installed over numerous items and areas. There is also a long-felt need in the art for a sanitizing scan device that allows the UV lights to kill germs, viruses, and bacteria prior to anyone touching the surface to help prevent the spread of illness. Further, there is a long-felt need in the art for a sanitizing scan device that reflects UV light within the system to fully cleanse the entire area of harmful bacteria. Moreover, there is a long-felt need in the art for a device that includes a motion sensor that expands and retracts a cover over the surface, ensuring it is only accessible when a person is ready to touch or use that surface. Further, there is a long-felt need in the art for a sanitizing scan device that improves public safety while reducing the amount of manual labor required to clean different high traffic surfaces. Finally, there is a long-felt need in the art for a sanitizing scan device that is powered by an internal battery or transformer for larger applications.

The present invention, in one exemplary embodiment, is a novel sanitizing scan device that utilizes ultraviolet (UV) light for eliminating germs, bacteria, and viruses from different surfaces. The sanitizing scan device comprises a housing and cover that is a durable, weather, insect, and water-proof gasket-sealed system. The housing is made from a durable plastic or other suitable material that also conceals the UV-C light during operation. Further, the housing comprises a motion-operated sensor mounted near the opening to the housing. The sensor recognizes the approach of a user's hand and acts to open the cover. Additionally, based on application size, the device is powered by an internal, rechargeable battery or transformer for larger applications. The present invention also includes a novel method of sanitizing a high-use item with UV lights to kill germs, viruses, and bacteria. The method includes the steps of providing a sanitizing scan device comprising a housing and cover with UV lights positioned in the interior of the housing for sanitation. The method also comprises positioning the housing over an item to be sanitized, such as a light switch. Further, the method comprises securing the cover over the housing and light switch. The method also comprises sanitizing the light switch via the UV light within the housing. Finally, the method comprises sensing a user's hand via the motion sensor to open the cover and expose the light switch for use.

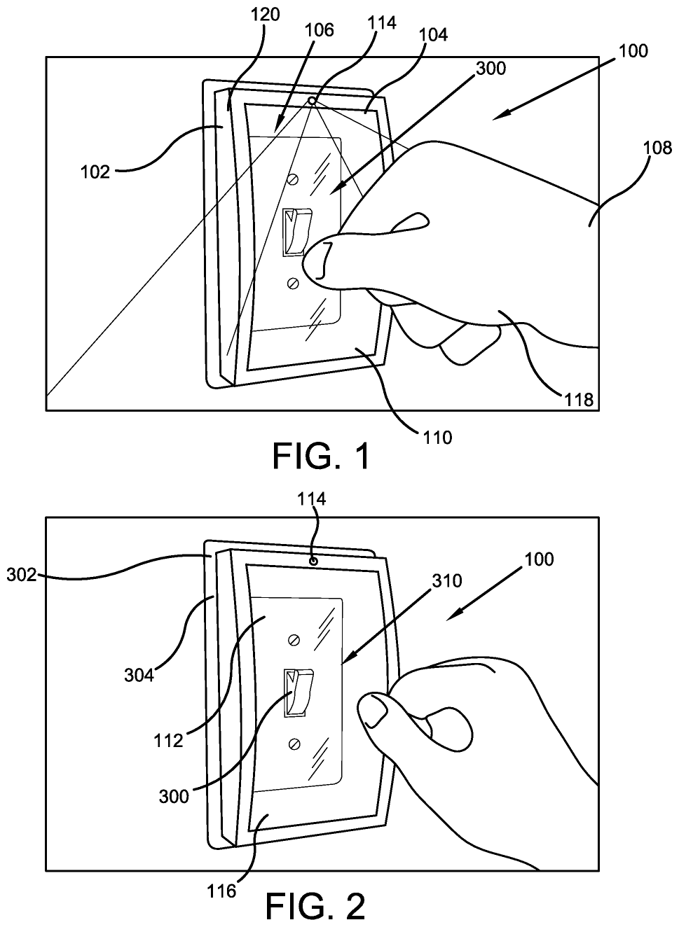

Referring initially to the drawings, FIGS. 1-2 illustrate a perspective view of one embodiment of the sanitizing scan device 100 of the present invention. In the present embodiment, the sanitizing scan device 100 is an improved sanitizing scan device 100 that provides a user 108 with a sanitization system that utilizes ultraviolet (UV) light 106 for eliminating germs, bacteria, and viruses from different surfaces. In use, the UV light 106 is refracted and covers the surface area to be sanitized. Specifically, the sanitizing scan device 100 comprises a housing 102 and cover 104 with a plurality of radiating sources 106 for sanitation. Further, size and configuration details of the housing 102 would vary according to application, but examples include gas pump handles, public bathroom stall handles 400, public hand dryers, light switches 300, residential and commercial entryways, and exits, appliances, vehicle handles, etc., or any other suitable objects or surfaces as is known in the art, depending on the needs and/or wants of a user 108.

Generally, the sanitizing scan device 100 is a UV light sanitization device that may be used to effectively and automatically disinfect contact surfaces, objects, and the like, and that can eliminate the disease-causing microbes commonly found thereon. Additionally, the sanitizing scan device 100 is easily and automatically controlled by a user 108 and prevents the transmission of harmful pathogens and therefore prevents the spread of infectious diseases. Moreover, the sanitizing scan device 100 is portable and relatively maintenance free, inexpensive to manufacture and operate, and is both safe and easy to use.

Furthermore, the sanitizing scan device 100 comprises a housing 102 with a cover 104 and a plurality of ultraviolet radiating sources 106 contained therein that emit light at the proper wavelength suitable for sanitizing the surrounding area. The emitted germicidal light is refracted and covers the surface area to be sanitized. Specifically, the interior 110 of the housing 102 is coated with a reflective durable material 112 that will ensure the UV light 106 covers the entire surface area within the housing 102.

Additionally, the ultraviolet radiating sources 106 can be high intensity ultraviolet lamps, ultraviolet light emitting diodes (LEDs), super luminescent LEDs, or a combination thereof. The UV radiating sources 106 are preferably UV-C, having a significant portion of light output in the region of 100 nm to 400 nm wavelength, preferably 200 to 300 nm wavelength, more preferably 250 nm to 260 nm wavelength. Further, ultraviolet light sources 106 can also be interchanged to provide additional types of radiation to the item. Typically, ultraviolet light sources 106 can produce light having wavelengths from about one hundred nanometers to about four hundred nanometers. More preferably, however, ultraviolet light 106 having wavelengths of about two hundred fifty nanometers have been used to eliminate microbes. In one embodiment, pulsating ultraviolet light sources 106 may be used. Experiments have shown that pulsating ultraviolet light sources more effectively kill bacteria and other microbes that affect the items to be sanitized. In preferred embodiments, ultraviolet light sources 106 can be made from quartz and a material called soft glass.

Furthermore, the housing 102 comprises a motion sensor 114 mounted near the front opening 116 of the housing 102. The motion sensor 114 acts to recognize the approach of a user's hand 118 or other body part. The motion sensor 114 can be any suitable sensor as is known in the art and can be positioned in any suitable area on the housing 102, as long as the motion sensor 114 is positioned to easily detect a user 108 and open the cover 104. Once the motion sensor 114 recognizes the user's hand 118, the cover 104 is automatically opened to allow access to the interior 110 of the housing 102, and thus utilize the light switch 300 or other object contained within. Accordingly, the cover 104 can be secured to the housing 102 via a movable hinge 120, telescoping cover, sliding cover, etc. Thus, the motion sensor 114 expands and retracts the cover 104 over the housing 102, ensuring the interior 110 of the housing 102 is only accessible when a person 108 is ready to touch or use the sanitized surface.

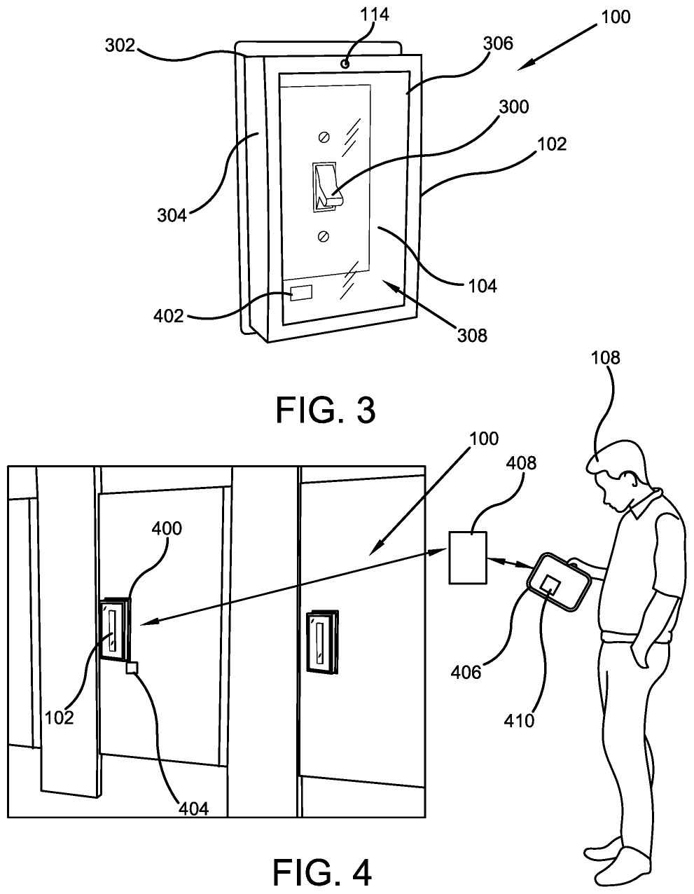

As shown in FIG. 3, the housing 102 is configured in a cylindrical, oval, square, or any other suitable shape as is known in the art, depending on the application. Typically, the housing 102 has an open back 302 and a perimeter sidewall 304 with an open front 116, creating a cavity (i.e., interior 110) for encompassing the object (i.e., light switch 300) to be sanitized. The open back 302 of the housing 102 is placed around the object 300 to be sanitized and secured in place via any suitable securing means. The cover 104 of the device 100 is then sealed to the front of the housing 102, completely enclosing the object 300. The housing 102 and cover 104 are made from a durable plastic or other suitable material that also conceals the UV-C light 106 during operation. The interior 110 of the housing 102 and cover 104 are also coated with a reflective durable material 112 that will ensure the UV light 106 covers the entire interior 110 surface area within the housing 102 and cover 104, to completely sanitize the enclosed object 300.

Furthermore, the cover 104 is preferably sealed to the housing 102 around the periphery via a gasket seal 306 and the rear surface 302 of the housing 102 has an adhesive tape, or other means, to mount the housing 102 around any surface or object 300 to be sanitized. Alternatively, the rear surface 302 may include slotted holes for mounting the device 100 to a wall using one or more screws or other fasteners. The adhesive tape at the rear surface 302 allows the scan device 100 to removably attach to a surface, and thus increases the utility of the scan device 100. Thus, the sanitizing scan device 100 provides effective protection from germs, viruses, bacteria, dust mites, and mold without the use of aerosol sprays or other harsh chemicals that may be harmful if inhaled by an individual, particularly someone with a respiratory illness.

Additionally, the interior sidewalls 304 of the housing 102 include reflective material 112 to redirect ultraviolet light from the ultraviolet radiating sources 106. The reflective material 112 can be made of a thin polished layer of metal 308, such as aluminum, which is deposited on glass substrates. Other reflective materials may include, but are not limited to, silver and gold. Silver, which has a reflectivity of about ninety-five percent to about ninety-nine percent can reflect ultraviolet spectral regions. Gold, which has a reflectivity of about ninety-eight percent to about ninety-nine percent, can reflect light which has wavelengths below five hundred fifty nanometers. Increasing and decreasing the density and thickness of the metals 308 used also determines the reflectivity of the sidewalls. Preferably, the metals 308 are grounded.

In one embodiment, instead of using reflective metals 308 as described above, reflective coatings 310 can be placed on the sidewalls 304. In exemplary embodiments, dielectric coatings 310 having a different refractive index to the substrate may be used. Dielectric coatings 310 can include magnesium fluoride, calcium fluoride, and various metal oxides, which are deposited onto a substrate. By carefully choosing the exact composition, thickness, and number of layers, reflectivity and transmitivity of the coating 310 can produce any desired characteristic.

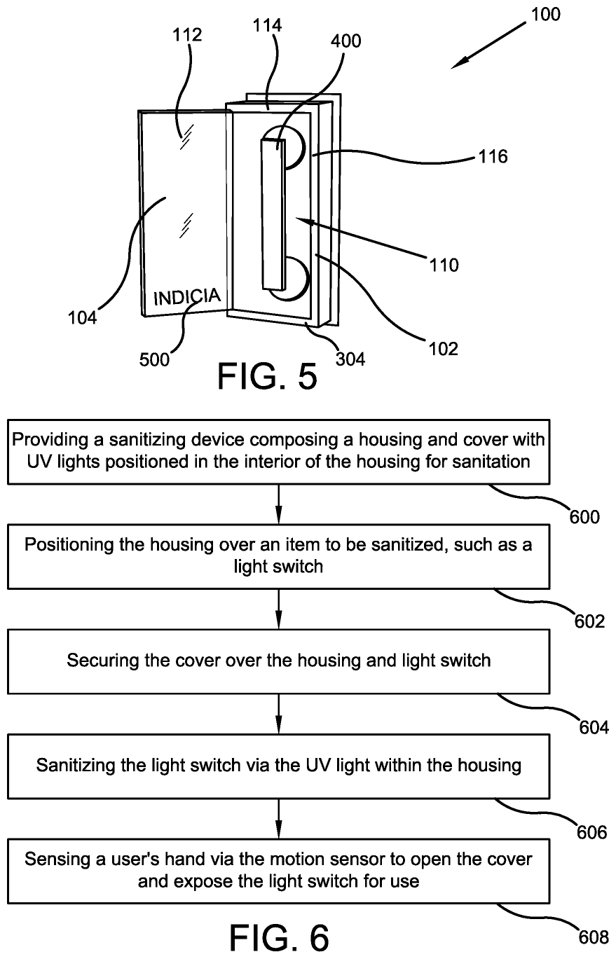

As shown in FIGS. 4-5, based on application size, the device 100 is powered via an internal, rechargeable battery 402 or transformer 404 for larger applications. Example applications include, but are not limited to, gas pump handles, public bathroom stall handles 400, public hand dryers, light switches 300, residential and commercial entryways and exits, appliances, vehicle handles, etc., or any other suitable objects or areas as is known in the art.

Furthermore, the housing 102 comprises a battery 402. The battery 402 may be a disposable battery or a rechargeable battery in the form of an alkaline, nickel-cadmium, nickel-metal hydride battery, etc., such as any 3V-12 volts DC battery or other conventional battery, such as A, AA, AAA, etc., that supplies power to the device 100. Throughout this specification, the term “battery” may be used interchangeably to refer to one or more wet or dry cells or batteries of cells in which chemical energy is converted into electricity and used as a source of DC power. References to recharging or replacing the battery 402 may refer to recharging or replacing individual cells, individual batteries of cells, or a package of multiple battery cells as is appropriate for any given battery technology that may be used. In addition, a rechargeable embodiment of the battery 402 may be recharged using a USB port (not shown), wherein the USB port is a USB-A, USB-B, Micro-B, Micro-USB, Mini-USB, or USB-C port, etc.

In one embodiment, the device 100 can be controlled by sending an instruction from the user 108 through a remote device 406, such as a smartphone, tablet, smart watch, computer, or the like. Thus, the sanitizing scan device 100 may be controlled wirelessly through a remote or electronic device 406. The remote or electronic device 406 may perform any type of wireless communication, including, but not limited to, WIFI, BLUETOOTH, RFID, NFC, etc. The device 100 further includes a wireless communication module 408 and additional sensors which would allow the device 100 to pair with a mobile application 410 on a remote or electronic device 406. Once paired, a user 108 could control the device 100 via the mobile application 410.

In yet another embodiment, the sanitizing scan device 100 comprises a plurality of indicia 500. The housing 102 of the device 100 may include advertising, a trademark, or other letters, designs, or characters, printed, painted, stamped, or integrated into the housing 102, or any other indicia 500 as is known in the art. Specifically, any suitable indicia 500 as is known in the art can be included, such as but not limited to, patterns, logos, emblems, images, symbols, designs, letters, words, characters, animals, advertisements, brands, etc., that may or may not be sanitation, scan, or brand related.

FIG. 6 illustrates a flowchart of the method of sanitizing a high-use item with UV lights to kill germs, viruses, and bacteria. The method includes the steps of at 600, providing a sanitizing scan device comprising a housing and cover with UV lights positioned in the interior of the housing for sanitation. The method also comprises at 602, positioning the housing over an item to be sanitized, such as a light switch. Further, the method comprises at 604, securing the cover over the housing and light switch. The method also comprises at 606, sanitizing the light switch via the UV light within the housing. Finally, the method comprises at 608, sensing a user's hand via the motion sensor to open the cover and expose the light switch for use.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “sanitizing scan device”, “sanitizing device”, “scan device”, and “device” are interchangeable and refer to the sanitizing scan device 100 of the present invention.

Notwithstanding the foregoing, the sanitizing scan device 100 of the present invention can be of any suitable size and configuration as is known in the art without affecting the overall concept of the invention, provided that it accomplishes the above-stated objectives. One of ordinary skill in the art will appreciate that the sanitizing scan device 100 as shown in FIGS. 1-6 is for illustrative purposes only, and that many other sizes and shapes of the sanitizing scan device 100 are well within the scope of the present disclosure. Although the dimensions of the sanitizing scan device 100 are important design parameters for user convenience, the sanitizing scan device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sanitizing scan device that utilizes ultraviolet (UV) light for eliminating germs, bacteria, and viruses from different surfaces, the sanitizing scan device comprising:
a housing;
a cover; and
a plurality of radiating sources;
wherein the housing is secured over an object to be sanitized;
wherein the cover is secured over the housing to fully enclose the object; and
further wherein the plurality of radiating sources are positioned within the housing and produce ultraviolet (UV) light to sanitize the object.

2. The sanitizing scan device of claim 1, wherein size of the housing varies according to application, which application includes gas pump handles, public bathroom stall handles, public hand dryers, light switches, residential and commercial entryways, and exits, appliances, or vehicle handles.

3. The sanitizing scan device of claim 2, wherein the plurality of ultraviolet radiating sources can be high intensity ultraviolet lamps, ultraviolet light emitting diodes (LEDs), super luminescent LEDs or a combination thereof.

4. The sanitizing scan device of claim 3, wherein the plurality of UV radiating sources are preferably UV-C and have a significant portion of light output in the region of 100 nm to 400 nm wavelength, preferably 200 to 300 nm wavelength, and more preferably 250 nm to 260 nm wavelength.

5. The sanitizing scan device of claim 4, wherein the plurality of UV radiating sources comprises pulsating ultraviolet light sources.

6. The sanitizing scan device of claim 5, wherein the housing comprises a motion sensor mounted near a front opening of the housing to sense approach of a user's hand, which opens the cover.

7. The sanitizing scan device of claim 6, wherein the housing has an open back and a perimeter sidewall with an open front, creating a cavity for encompassing the object to be sanitized.

8. The sanitizing scan device of claim 7, wherein the open back of the housing is placed around the object to be sanitized and secured in place via adhesive tape and the cover is then sealed to a front of the housing via a gasket seal.

9. The sanitizing scan device of claim 8, wherein the housing and cover are made from a durable plastic that conceals the UV-C light during operation.

10. The sanitizing scan device of claim 9, wherein the interior of the housing and cover are coated with a reflective durable material that will ensure UV light covers entire interior surface area within the housing and cover, to completely sanitize enclosed object.

11. The sanitizing scan device of claim 10, wherein the sanitizing scan device is powered by a rechargeable battery.

12. The sanitizing scan device of claim 10 wherein the sanitizing scan device is powered by a transformer.

13. A sanitizing scan device that utilizes ultraviolet (UV) light for eliminating germs, bacteria, and viruses from different surfaces, the sanitizing scan device comprising:
a housing comprising an open back and a perimeter sidewall with an open front, creating a cavity for encompassing an object to be sanitized;
a cover secured over the housing to fully enclose the object; and
a plurality of radiating sources that are preferably UV-C and have a significant portion of light output in the region of 100 nm to 400 nm wavelength, preferably 200 to 300 nm wavelength, and more preferably 250 nm to 260 nm wavelength;
wherein the open back of the housing is placed around the object to be sanitized and secured in place via adhesive tape and the cover is then sealed to a front of the housing via a gasket seal;
wherein the housing and cover are made from a durable plastic that conceals the UV-C light during operation;
wherein the housing comprises a motion sensor mounted near a front opening of the housing to sense approach of a user's hand, which automatically opens the cover;
wherein the plurality of radiating sources are positioned within the housing and produce UV-C light to sanitize the object;
wherein the interior of the housing and cover are coated with a reflective durable material that will ensure the UV-C light covers entire interior surface area within the housing and cover, to completely sanitize enclosed object; and
further wherein the sanitizing scan device is powered by a rechargeable battery or transformer.

14. The sanitizing scan device of claim 13, wherein interior sidewalls of the housing include reflective material, such as a thin polished layer of metal, to redirect ultraviolet light from the plurality of ultraviolet radiating sources.

15. The sanitizing scan device of claim 13, wherein interior sidewalls of the housing include reflective coatings, such as dielectric coatings having a different refractive index to a substrate, to redirect ultraviolet light from the plurality of ultraviolet radiating sources.

16. The sanitizing scan device of claim 13, wherein the sanitizing scan device is controlled wirelessly through a remote device, such as a smartphone, tablet, smart watch, or computer.

17. The sanitizing scan device of claim 16 further comprising a wireless communication module and additional sensors which would allow the sanitizing scan device to pair with a mobile application on the remote device.

18. The sanitizing scan device of claim 13 further comprising a plurality of indicia.

19. The sanitizing scan device of claim 13, wherein size of the housing varies according to application, which application includes gas pump handles, public bathroom stall handles, public hand dryers, light switches, residential and commercial entryways, and exits, appliances, or vehicle handles.

20. A method of sanitizing a high-use item with UV lights to kill germs, viruses, and bacteria, the method comprising the following steps:
providing a sanitizing scan device comprising a housing and cover with UV lights positioned in the interior of the housing for sanitation;
positioning the housing over an item to be sanitized, such as a light switch;
securing the cover over the housing and light switch;
sanitizing the light switch via the UV light within the housing; and
sensing a user's extremity via the motion sensor to open the cover and expose the light switch for use.
